(19) United States
(12) Patent Application Publication
(10) Pub. No.: US 2022/0218236 A1
(43) Pub. Date: Jul. 14, 2022

(54) Systems and Methods for Hearing Evaluation

(71) Applicant: SONOVA AG, Staefa (CH)

(72) Inventors: Olaf Strelcyk, Cincinnati, OH (US); Charlotte Vercammen, Zürich (CH); Nicola Hildebrand, Uster (CH)

(73) Assignee: Sonova AG

(21) Appl. No.: 17/148,138

(22) Filed: Jan. 13, 2021

(51) Int. Cl. A61B 5/12; G06N 20/00; G06F 17/18

(52) U.S. Cl. CPC A61B 5/121; G06F 17/18; G06N 20/00

(57) ABSTRACT

An exemplary hearing evaluation system is configured to obtain a first auditory measurement dataset representative of one or more auditory measurements corresponding to a first ear of a user and that are acquired using a first auditory measurement test procedure having a first acoustic stimulus attribute set, obtain a second auditory measurement dataset representative of one or more auditory measurements corresponding to a second ear of the user and that are acquired using a second auditory measurement test procedure having a second acoustic stimulus attribute set different than the first acoustic stimulus attribute set, obtain a questionnaire response dataset representative of one or more responses to one or more question items associated with the user, and determine, based on the first auditory measurement dataset, the second auditory measurement dataset, and the questionnaire response dataset, a hearing profile of the user.

### BACKGROUND INFORMATION

To determine whether a user is a good candidate for a hearing device (e.g., a hearing aid), a hearing care professional may perform a diversity of auditory tests configured to determine an individualized hearing profile for the user. The hearing profile may indicate a hearing ability of the user and/or how the user might respond to the hearing device. If the user decides to get a hearing device, the hearing profile may, in some instances, be used to fit (e.g., program one or more settings of) the hearing device to the user.

It is often inconvenient or impossible for a user to meet with a hearing care professional in person so that the hearing care professional may evaluate the user's hearing. Accordingly, various attempts have been made to allow for self-administered hearing evaluations that can be performed by users in any location via online testing. Unfortunately, these self-administered hearing evaluations are susceptible to a variety of errors and/or uncertainties. For example, deviations and/or errors in sound spectra, sound pressure levels, signal-to-noise ratios, and/or signal-to-background ratios delivered at the user's ears or eardrums may be introduced by use of unknown and uncontrolled devices and equipment. Moreover, the use of wrong sound volume or frequency equalizer device settings, environmental noise, and user error (e.g., the user mixing up left ear and right ear sound transducers) can often make self-administered hearing evaluations inaccurate and unreliable.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary hearing evaluation system.

FIGS. 2-3 illustrate exemplary configurations of the hearing evaluation system of FIG. 1.

FIG. 4 shows a configuration in which an input module is in communication with an estimation module.

FIGS. 5-7 show configurations in which an estimation module uses a predictive model to generate hearing profile data representative of a hearing profile for the user.

FIG. 8 shows a configuration in which an estimation module is implemented by multiple processing stages.

FIG. 9 shows a configuration in which feedback data generated by an estimation module is configured to adaptively influence an operation of an input module.

FIG. 10 shows a configuration in which an output module is configured to generate output data based on hearing profile data output by an estimation module.

FIG. 11 shows a configuration in which a quality estimation module is configured to estimate a quality of a hearing profile.

FIG. 12 shows a configuration in which the hearing evaluation system of FIG. 1 obtains a device dataset and uses the device dataset to assist in determining a hearing profile for a user.

FIG. 13 shows a configuration in which the hearing evaluation system of FIG. 1 obtains a user profile dataset and uses the user profile dataset to assist in determining a hearing profile for a user.

FIG. 14 shows a configuration in which a fitting system is configured to use hearing profile data to generate fitting data that may be used to fit a hearing device to a user.

FIG. 15 shows a graph that shows how the systems and methods described herein may be used to determine a hearing profile for a user.

FIG. 16 illustrates an exemplary method.

FIG. 17 illustrates an exemplary method.

FIG. 18 illustrates an exemplary computing device.

### DETAILED DESCRIPTION

Systems and methods for hearing evaluation are described herein. For example, as described more fully herein, an illustrative hearing evaluation system may be configured to obtain a first auditory measurement dataset representative of one or more auditory measurements corresponding to a first ear of a user and that are acquired using a first auditory measurement test procedure having a first acoustic stimulus attribute set, obtain a second auditory measurement dataset representative of one or more auditory measurements corresponding to a second ear of the user and that are acquired using a second auditory measurement test procedure having a second acoustic stimulus attribute set different than the first acoustic stimulus attribute set, obtain a questionnaire response dataset representative of one or more responses to one or more question items associated with the user, and determine, based on the first auditory measurement dataset, the second auditory measurement dataset, and the questionnaire response dataset, a hearing profile of the user.

As used herein, a hearing profile for a user may include an audiogram and/or any other profile that indicates a likelihood of hearing loss or hearing impairment in one or both ears of the user. For example, the hearing profile may be representative of binaural hearing impairment, where such hearing impairment does not just reflect hearing loss in terms of elevated pure-tone thresholds but also supra-threshold deficits such as caused by hidden hearing loss. As another example, the hearing profile may be determined as a function of both frequency and ear (e.g., pure-tone threshold estimates for individual frequencies and ears).

By using auditory measurement datasets together with responses to question items associated with the user to determine the hearing profile, the systems and methods described herein may be more robust to errors and uncertainties compared to conventional hearing evaluation approaches. The systems and methods may accordingly facilitate accurate and effective self-administered hearing evaluations, which may allow for users to select and be fitted with a hearing device from the comfort of their own homes.

Moreover, by using different acoustic stimulus attribute sets for each auditory measurement test procedure used for the different ears of a user, the systems and methods may allow for a more efficient and thorough evaluation of a user's hearing capability compared to conventional approaches that use the same acoustic stimulus attribute set for each ear. For example, by using a first set of distinct frequencies (e.g., relatively low frequencies) for the acoustic stimuli presented to the first ear and a second set of distinct frequencies (e.g., relatively high frequencies) for the acoustic stimuli presented to the second ear, and then applying the results to a predictive model as described herein, the systems and methods described herein may yield information about ear asymmetries without the user having to take the time to use the same frequency sets for both ears. As another example, by using consonant identification in one ear and vowel identification in the other ear, the systems and methods described herein provide the benefit of using two different speech materials, while also yielding information about ear asymmetries (both at the same time, which is more efficient than measuring both speech materials in both ears).

As used herein, a hearing device may be implemented by any device configured to provide hearing assistance to a user. For example, a hearing device may be implemented by a hearing aid configured to amplify audio content to a user, a sound processor included in a cochlear implant system configured to apply electrical stimulation representative of audio content to a user, a sound processor included in a stimulation system configured to apply electrical and acoustic stimulation to a user, an assisted listening device, and/or any other suitable hearing prosthesis or combination of hearing prostheses.

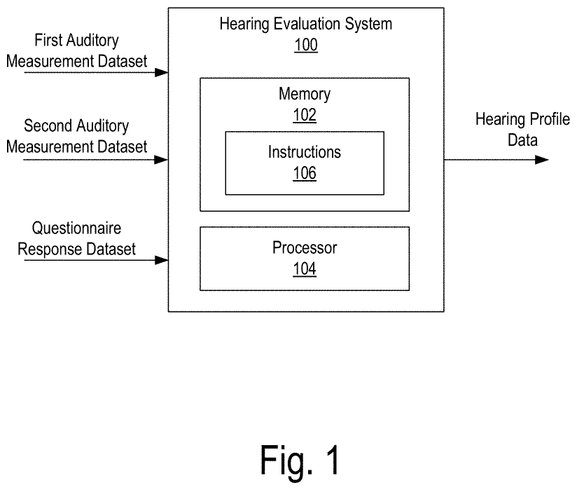

FIG. 1 illustrates an exemplary hearing evaluation system 100 (“system 100”). As shown, system 100 may include, without limitation, a memory 102 and a processor 104 selectively and communicatively coupled to one another. In some embodiments, memory 102 and processor 104 may be distributed between multiple devices and/or multiple locations as may serve a particular implementation.

In some embodiments, memory 102 may be implemented by any suitable non-transitory computer-readable medium and/or non-transitory processor-readable medium, such as any combination of non-volatile storage media and/or volatile storage media as described herein. In some embodiments, memory 102 may maintain (e.g., store) executable data used by processor 104 to perform one or more operations of system 100 described herein. For example, memory 102 may store instructions 106 that may be executed by processor 104 to perform any of the operations associated with system 100 described herein. Instructions 106 may be implemented by any suitable application, software, code, and/or other executable data instance.

In some embodiments, memory 102 may also maintain any data generated, managed, used, transmitted, and/or received by processor 104. For example, memory 102 may maintain any of the datasets described herein, data representative of a predictive model used to determine a hearing profile for a user, and/or any other suitable data.

Processor 104 may be configured to execute instructions 106 to perform various operations described herein as being performed by system 100. For example, as illustrated in FIG. 1, system 100 (e.g., processor 104) may obtain a first auditory measurement dataset representative of one or more auditory measurements corresponding to a first ear of a user and that are acquired using a first auditory measurement test procedure having a first acoustic stimulus attribute set, obtain a second auditory measurement dataset representative of one or more auditory measurements corresponding to a second ear of a user and that are acquired using a second auditory measurement test procedure having a second acoustic stimulus attribute set different than the first acoustic stimulus attribute set and obtain a questionnaire response dataset representative of one or more responses to one or more question items associated with the user. System 100 may be further configured to determine, based on the first auditory measurement dataset, the second auditory measurement dataset, and the questionnaire response dataset, a hearing profile of the user and output hearing profile data representative of the hearing profile. These and other operations that may be performed by system 100 are described herein.

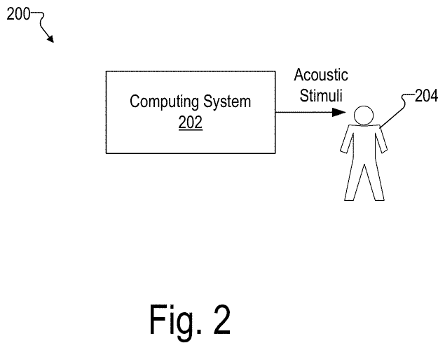

FIG. 2 shows a configuration 200 in which system 100 is implemented by a computing system 202 located within a vicinity of a user 204 for whom a hearing evaluation is to be performed. Computing system 202 may be implemented by one or more computing devices, such as one or more desktop computers, mobile devices (e.g., mobile phones), portable computers, specialized testing equipment, etc.

Computing system 202 may be configured to perform one or more auditory measurement test procedures with respect to user 204 to obtain the first and second auditory measurement datasets described herein. To this end, as shown, computing system 202 may be configured to apply acoustic stimuli to the user 204 (e.g., a first set of acoustic stimuli to a first ear of user 204 and a second set of acoustic stimuli to a second ear of user 204). Computing system 202 may apply acoustic stimuli to user 204 in any suitable manner. For example, the acoustic stimuli may be presented to user 204 by way of one or more sound transducers (e.g., loudspeakers, headphones, and/or earphones) connected to or included in computing system 202.

Computing system 202 may be further configured to collect data representative of one or more responses of user 204 to the acoustic stimuli. The collected data may be included in the auditory measurement datasets described herein.

In some examples, computing system 202 is located within a hearing care professional premises (e.g., a clinic, office, or other location) associated with a hearing care professional (e.g., a clinician, a doctor, etc.) that specializes in performing hearing evaluations with respect to users. In these examples, user 204 may travel to the hearing care professional premises so that the hearing evaluation may be performed on user 204 in person by the hearing care professional.

Alternatively, computing system 202 may be located remote from a hearing care professional premises. For example, computing system 202 may be located within a home or other user premises associated with the user. In these configurations, user 204 may use computing system 202 to perform a self-administered hearing evaluation with or without the aid of a hearing care professional.

To illustrate, computing system 202 may be implemented by a mobile device that executes a mobile application configured to perform a hearing evaluation with respect to user 204. For example, the mobile application may be configured to present acoustic stimuli to user 204 by way of one or more sound transducers connected to or included in the mobile device. The mobile application may be further configured to collect data representative of one or more responses by user 204 to the acoustic stimuli. The mobile application may be further configured to process and/or transmit the collected data to another system for processing. This is described in more detail herein.

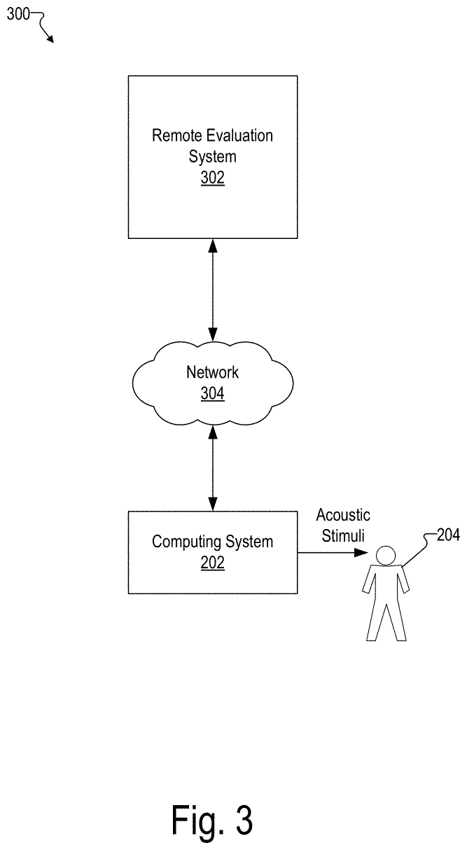

FIG. 3 shows an exemplary configuration 300 in which system 100 is at least partially implemented by a remote evaluation system 302 communicatively coupled to computing system 202 by way of a network 304. Network 304 may be implemented by the Internet, a wide area network, a local area network, a wireless network (e.g., Wi-Fi), a cellular data network, and/or any other suitable network. Data may flow between components connected to network 304 using any communication technologies, devices, media, and protocols as may serve a particular implementation.

In some examples, system 100 is entirely implemented by remote evaluation system 302. In these examples, system 100 may obtain the datasets described herein by receiving the datasets from computing system 202 and/or any other computing device by way of network 304. Alternatively, system 100 may be implemented by a combination of remote evaluation system 302 and computing system 202.

Remote evaluation system 302 may be implemented by any suitable combination of one or more computing devices. For example, remote evaluation system 302 may be implemented by one or more servers. In some examples, remote evaluation system 302 may be associated with (e.g., provided and/or maintained by) a hearing care professional, an entity that specializes in providing remote hearing evaluation capability, and/or any other suitable entity is may serve a particular implementation.

As described herein, remote evaluation system 302 may be configured to direct computing system 202 to perform an auditory measurement test with respect to user 204. For example, remote evaluation system 302 may transmit a command by way of network 304 to computing system 202 for computing system 202 to present auditory stimuli to one or both ears of user 204. Remote evaluation system 302 may be further configured to direct computing system 202 to present one or more question items to the user and/or to a different user (e.g., a parent of the user). Upon acquiring data representative of one or more responses by user 204 to the acoustic stimuli and data representative of one or more responses to the question items, computing system 202 may transmit the acquired data to remote evaluation system 302, which may process the acquired data to generate a hearing profile for user 204 in any of the ways described herein.

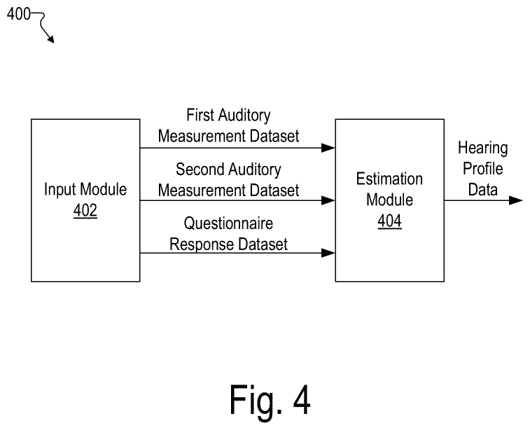

FIG. 4 illustrates exemplary configuration 400 that includes an input module 402 in communication with an estimation module 404. Modules 402 and 404 may include any suitable combination of hardware and/or software and may be implemented by any of the systems described herein (e.g., by system 100, remote evaluation system 302, and/or computing system 202).

Input module 402 is configured to acquire the first and second auditory measurement datasets and the questionnaire response dataset as described herein. Estimation module 404 is configured to use the acquired datasets to generate hearing profile data representative of a hearing profile of a user. Exemplary manners in which input module 402 and estimation module 404 may perform these operations are described herein.

In some examples, input module 402 may acquire the first auditory measurement dataset corresponding to the first ear of the user by performing a first auditory measurement test procedure with respect to the first ear of the user. The first auditory measurement test procedure has a first acoustic stimulus attribute set. For example, input module 402 may direct an acoustic signal generator (e.g., a component within or connected to computing system 202) to present one or more auditory stimuli having one or more attributes included in the first acoustic stimulus set to the first ear of the user. Input module 402 may then measure one or more responses by the user to the one or more auditory stimuli.

Likewise, input module 402 may acquire the second auditory measurement dataset corresponding to the second ear of the user by performing a second auditory measurement test procedure with respect to the second ear of the user. The second auditory measurement test procedure has a second acoustic stimulus attribute set. For example, input module 402 may direct an acoustic signal generator (e.g., a component within or connected to computing system 202) to present one or more auditory stimuli having one or more attributes included in the second acoustic stimulus set to the second ear of the user. Input module 402 may then measure one or more responses by the user to the one or more auditory stimuli.

Auditory measurements performed by input module 402 may be free-field, monaural, diotic, dichotic, and/or any other type of measurements involving auditory stimuli delivered to the user via one or more sound transducers, such as loudspeakers, headphones, or earphones. The auditory measurements can be threshold and supra-threshold measurements. These can be (but are not limited to) measurements of detection thresholds, most comfortable levels, and/or uncomfortable levels for various sounds such as tones, narrowband noises, speech, and/or bird song. Furthermore, the auditory measurements can be measurements of tone-in-noise detection thresholds, measurements of intelligibility of speech tokens such as phonemes, digits, or words in quiet or in various backgrounds such as interfering noises or talkers.

In some examples, auditory measurements involve a user responding to a presented auditory stimulus. The measurement procedure can be constant-stimuli, a method of adjustment, a method of limits, or adaptive such as an adaptive staircase procedure. The response can be given consciously or unconsciously. For example, the response can be given by manual input, by verbal response, and/or in any other suitable manner. A response may, for example, be recorded by video of the user such as pupillometry, by recording changes in electrical potential on the user's scalp (e.g., EEG), by recording the user's skin conductance, by brain sensors or other biosensors such as transdermal microneedles, optical sensors, and/or mechanical sensors (e.g., accelerometers).

As used herein, an acoustic stimulus attribute set of an auditory measurement test procedure refers to a set of attributes associated with auditory stimuli that is presented to a user during the auditory measurement test procedure. For example, an acoustic stimulus attribute set may include attributes representative of discrete frequencies of the auditory stimuli, spectral characteristics of the auditory stimuli, temporal characteristics of the auditory stimuli, perceptive attributes of the auditory stimuli, etc.

In some examples, the second acoustic stimulus attribute set of the second auditory measurement test procedure for the second ear is different than the first acoustic stimulus attribute set of the first auditory measurement test procedure for the second ear. In other words, the second acoustic stimulus attribute set includes one or more attributes not included in the first acoustic stimulus attribute set.

To illustrate, the first acoustic stimulus attribute set may include a first discrete frequency of a first acoustic stimulus (meaning that the first acoustic stimulus has or is centered at the first discrete frequency) presented to the user during the first auditory measurement test procedure. However, the second acoustic stimulus attribute set may include a second discrete frequency of a second acoustic stimulus presented to the user during the second auditory measurement test procedure, where the second discrete frequency is different than the first discrete frequency.

For example, the user input module may measure detection thresholds in dB HL for tones with various frequencies in the two ears. Such tones may be 1000-Hz tones and 6000-Hz tones in the left ear (IMmeas_{f=1000,e=L} and IMmeas_{f=6000,e=L}) and 2000-Hz tones and 4000-Hz tones in the right ear (IMmeas_{f=2000,e=R} and IMmeas_{f=4000,e=R}). In these equations, IM represents input module 402, f represents frequency, and e represents either the left (L) ear or the right (R) ear.

Advantages and benefits of having different acoustic stimulus attribute sets for the first and second auditory measurement test procedures are described herein.

Input module 402 may acquire the questionnaire response dataset in any suitable manner. For example, input module 402 may present, by way of a graphical user interface (e.g., a graphical user interface displayed by computing system 202), one or more questions items (or simply “questions”). The user may provide user input representative of one or more responses to the questions by, for example, interacting with the graphical user interface. Additionally or alternatively, input module 402 may audibly present the one or more questions, and the user input may be provided verbally by the user.

In some examples, at least one of the questions may be responded to by a person other than the user that is the subject of the hearing evaluation. For example, a family member (e.g., a parent), a teacher, a friend, and/or anyone else associated with the user may respond to one or more questions about the user. Such questions may include, but are not limited to, questions about the user's hearing status, questions about the user's hearing habits (e.g., television listening sound levels), questions about the user's speech production (e.g., voice levels produced by the user in quiet or noisy settings), questions about the measurement devices and equipment used in the auditory measurements, etc.

In these examples, the questions may be presented to the person by way of the same computing device (e.g., computing system 202) being used by the user and/or a different computing device (e.g., a mobile device associated with the person).

Any suitable number of user-related questions may be presented by input module 402. For example, input module 402 may present questions regarding personal details (e.g., the user's age or age decade, gender, occupation, hearing, general health status, etc.), details regarding personal assistive devices used by the user, details regarding the specific device and/or equipment used to perform the auditory measurement test procedures, etc.

For example, input module 402 may collect the user's responses to question items such as the following:

Question item 1: “Do you feel you have hearing issues?” Possible user responses include “No” (1), “Not sure” (2), or “Yes” (3).

Question item 2: “Do you find it hard to have a conversation on the phone?” Possible user responses include “Always” (1), “Often” (2), “Sometimes” (3), “Rarely” (4), or “Never” (5).

Question item 3: Do you find it hard to hear high-pitched sounds like bird song? Possible user responses include “Always” (1), “Often” (2), “Sometimes” (3), “Rarely” (4), or “Never” (5).

In some examples, one or more of the question items may be associated in a probabilistic sense with hearing health, hearing status, and/or hearing difficulties. Additionally or alternatively, one or more of the question items may differentiate between global, frequency-specific, ear-specific, peripheral auditory, and/or central auditory aspects of hearing. For example, the above question items about difficulties experienced with conversations on the phone or hearing high-pitched sounds are to some extent frequency-specific. Other examples are question items about ear-specific problems, such as indicating the extent of hearing difficulties in the better versus worse ear and/or indicating the presence of tinnitus in the ears. Other examples are question items that differentiate between degrees of self-perceived impact of hearing loss on quality of life (e.g. questions regarding social participation, cognitive health, etc.).

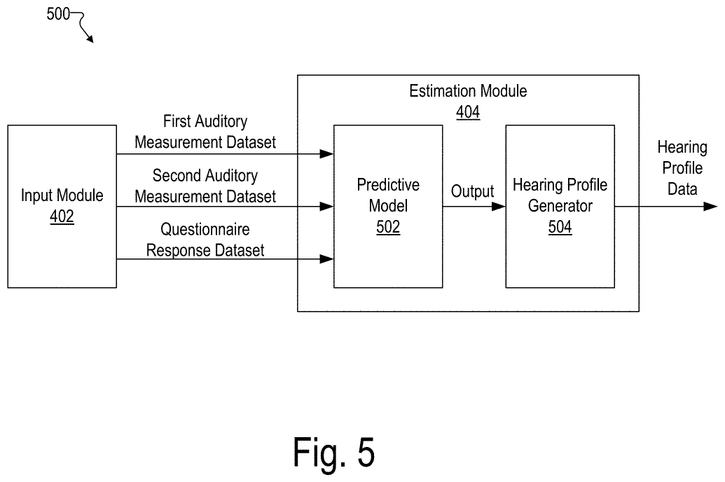

FIG. 5 shows a configuration 500 in which estimation module 404 uses a predictive model 502 to generate hearing profile data representative of a hearing profile for the user. For example, as shown, estimation module 404 may apply the first auditory measurement dataset, the second auditory measurement dataset, and the questionnaire response dataset as inputs to a predictive model 502. Based on an output of predictive model 502, a hearing profile generator 504 of estimation module 404 generates the hearing profile data. While hearing profile generator 504 is shown to be separate from predictive model 502, it will be recognized that in some examples, hearing profile generator 504 may be included in predictive model 502 such that predictive model 502 is configured to generate the hearing profile data.

Data representative of predictive model 502 may be maintained by one or more computing devices that implement system 100. For example, data representative of predictive model 502 may be stored locally by computing system 202 and/or remotely by a remote evaluation system 302. Additionally or alternatively, data representative of predictive model 502 may be maintained by a third-party system separate from any of the computing devices that implement system 100. In these configurations, estimation module 404 may transmit, by way of a network, the input datasets to the third-party system for processing by the predictive model 502 and receive output data from predictive model 502 by way of the network.

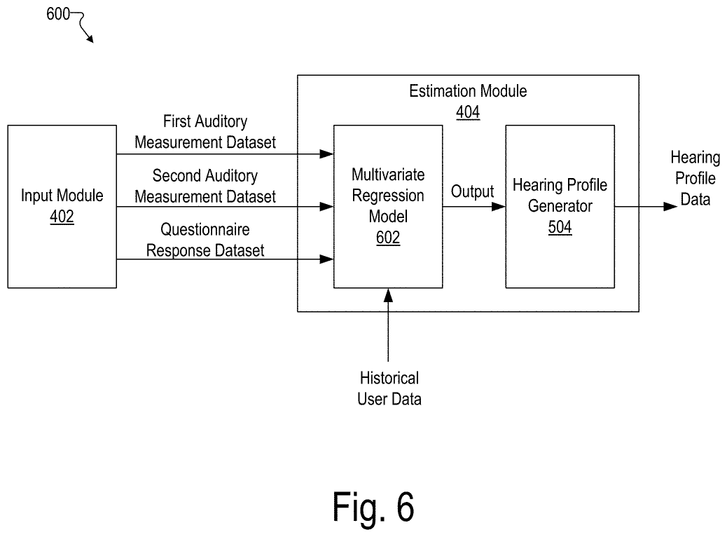

FIG. 6 shows an exemplary configuration 600 in which predictive model 502 is implemented by a multivariate regression model 602 that uses linear and/or nonlinear regression to generate an output. As illustrated, the output of multivariate regression model 602 may be based on a fitting of one or more regression models to historical user data corresponding to a plurality of users other than the user, where the historical user data is representative of auditory measurements performed on the plurality of users.

To illustrate, based on auditory measurement dataset values of tone detection thresholds in dB HL (IMmeas_{f,e}) and on responses to three question items (IMitemₘ, m=1, 2, 3) coded as numerals, the user's air-conduction pure-tone hearing thresholds in the left ear at 1000 Hz (HT_{f=1000,e=L}) and 6000 Hz (HT_{f=6000,e=L}) and in the right ear at 2000 Hz (HT_{f=2000,e=R}) and 4000 Hz (HT_{f=4000,e=R}) may be predicted by the following equations:

HT_{f=1000,e=L}=c_{1,0}+c_{1,1}*IMmeas_{f=1000,e=L}\hat{ }p_{1,1}+c_{1,2}*IMitem₁\hat{ }p_{1,2}+c_{1,3}*IMitem₂\hat{ }p_{1,3},

HT_{f=2000,e=R}=c_{2,0}+c_{2,1}*IMmeas_{f=2000,e=R}\hat{ }p_{2,1}+c_{2,2}*IMitem₁\hat{ }p_{2,2}+c_{2,3}*IMitem₃\hat{ }p_{2,3},

HT_{f=4000,e=R}=c_{4,0}+c_{4,1}*IMmeas_{f=4000,e=R}\hat{ }p_{4,1}+c_{4,2}*IMitem₁\hat{ }p_{4,2}+c_{4,3}*IMitem₃\hat{ }p_{4,3}, and

HT_{f=6000,e=L}=c_{6,0}+c_{6,1}*IMmeas_{f=6000,e=L}\hat{ }p_{6,1}+c_{6,2}*IMitem₁\hat{ }p_{6,2}+c_{6,3}*IMitem₃\hat{ }p_{6,3}.

In these equations, c_{n,k} are real-number constants and p_{n,k} are integer powers. In general, the best set of predictors for the air-conduction pure-tone hearing threshold at any given frequency can be determined by fitting regression models to user data from a sufficiently large group of users, for whom air-conduction pure-tone hearing thresholds measured according to a standard method are known, and by applying model selection techniques. In particular, in contrast to the above example equations, the input-module detection threshold measured for a tone or narrowband noise centered at a given frequency can also serve as a predictor of air-conduction pure-tone thresholds at other frequencies. The model parameters c_{n,k} and p_{n,k} may be derived, for example, using maximum likelihood estimation procedures. In addition, global, parameterwise, or joint shrinkage factors can be applied to the regression model coefficients to produce more robust estimates in terms of decreased prediction errors.

In some examples, multivariate regression model 602 may also contain interaction terms between the various predictor variables.

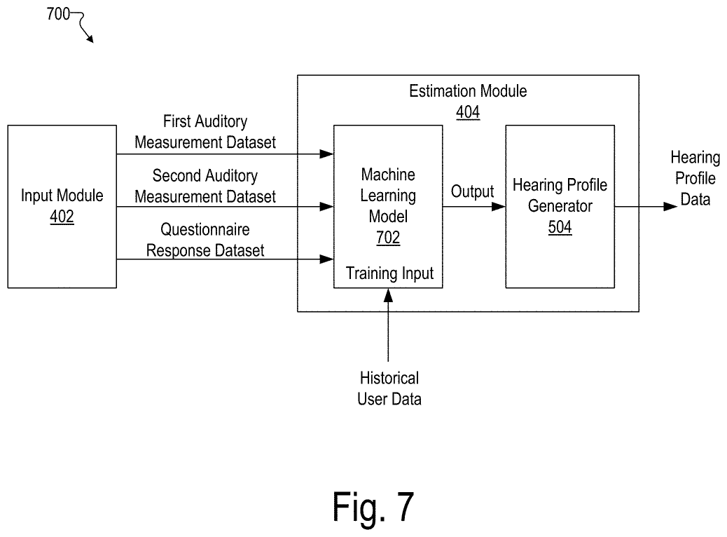

FIG. 7 shows an exemplary configuration 700 in which predictive model 502 is implemented by a machine learning model 702. As illustrated, machine learning model 702 may receive historical user data as a training input. In this manner, the historical user data may be used to train machine learning model 702. The historical user data may correspond to a plurality of users other than the user and may be representative of auditory measurements performed on the plurality of users, responses to one or more question items associated with the plurality of users, and/or any other data associated with the plurality of users as may serve a particular implementation.

As illustrated, the output of multivariate regression model 702 may be based on a fitting of one or more regression models to historical user data corresponding to a plurality of users other than the user, where the historical user data is representative of auditory measurements performed on the plurality of users.

Machine learning model 702 may be implemented by any supervised and/or unsupervised learning algorithms. For example, machine learning model 702 may be implemented by a supervised deep learning model, such as a neural network, a convolutional neural network, and/or a recurrent neural network.

In some examples, the hearing profile may be estimated by estimation module 404 in terms of other than air-conduction pure-tone hearing thresholds. For example, a logistic-regression model could be used to predict the likelihood of the presence of a hearing loss or hearing impairment. Hearing loss here may refer to elevated pure-tone hearing thresholds relative to a normal-hearing reference. Hearing impairment here may refer to a general degradation of hearing that can be present even for a user with a clinically normal audiogram. Hearing impairment could, for example, be quantified by way of a questionnaire, such as the hearing handicap inventory for adults.

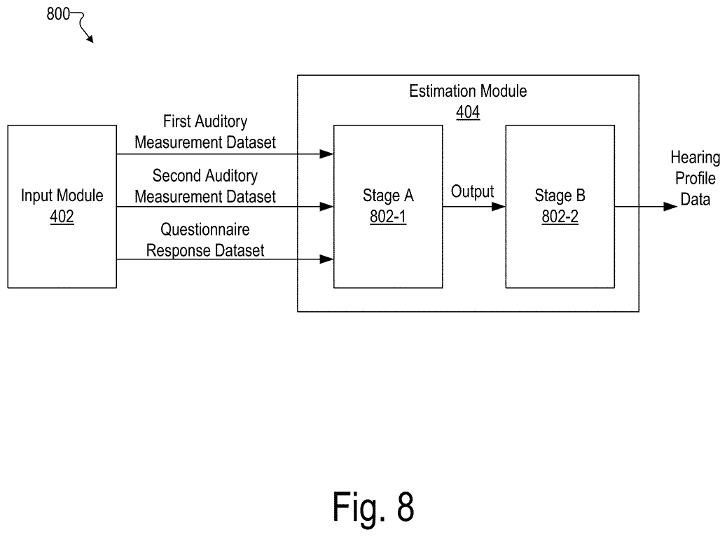

FIG. 8 shows a configuration 800 in which estimation module 404 is implemented by multiple processing stages. In particular, estimation module 404 is implemented by a first processing stage 802-1 (Stage A) and a second processing stage 802-2 (stage B). While two stages 802 are shown in FIG. 8, any number of processing stages may be used to generate hearing profile data.

To illustrate, processing stage 802-1 may correspond to one of the regression models described herein. The output of this stage are predicted pure-tone thresholds, such as HT_{f=1000,e=L}, HT_{f=2000,e=R}, HT_{f=4000,e=R}, and HT_{f=6000,e=L}. In this example, these outputs are referred to as meta thresholds MT_{f,e}, since they do not represent the final air-conduction pure-tone hearing threshold estimate of estimation module 404. Instead, these meta thresholds are used as input to processing stage 802-2, which produces the final estimated hearing profile.

Processing stage 802-2 may accomplish this by comparing the meta thresholds to a set of bilateral pure-tone audiogram prototypes that is representative of one or more user profile attributes of the user (e.g., the user's age and gender in the population at large). Processing stage 802-2 may select an audiogram prototype that most closely conforms to the predicted meta thresholds. This most likely audiogram prototype, consisting of left-ear and right-ear pure-tone hearing thresholds as a function of frequency, is the hearing profile estimate returned by estimation module 404.

In some examples, a set of k pure-tone audiogram prototypes that is representative of the user's age and gender in the population at large may, for example, be derived as follows. Starting with a large set of bilateral audiograms that constitute a representative sample of all audiograms in the population at large, all audiograms that correspond to the subgroup of people that match the user's age (e.g., age in decades) and gender may be selected. By applying a k-means clustering algorithm to these audiograms, the k pure-tone audiogram prototypes may be obtained as cluster centers. The audiogram prototype Aₙ that most closely conforms to the set of predicted meta thresholds SMT (in this example: SMT={MT_{f=1000,e=L}, MT_{f=2000,e=R}, MT_{f=4000,e=R}, MT_{f=6000,e=L}}) can be derived as the prototype that maximizes the unnormalized posterior Pᵤ(Aₙ|SMT):

Pu(Aₙ|SMT)=P(SMT|Aₙ)*P(Aₙ)=P(MT_{f=1000,e,L}|Aₙ)*P(MT_{f=2000,e=R}|Aₙ)*P(MT_{f=4000,e=R}|Aₙ)*P(MT_{f=6000,e=L}|Aₙ)*P(Aₙ).

In this equation, the informative prior P(Aₙ) is given as the proportion of all audiograms in the subgroup (of people that match the user's age and gender) that belong to the cluster of prototype Aₙ. Each term P(MT_{f,e}|Aₙ) represents the likelihood of the user producing a tone threshold of value MT_{f,e} given that his or her audiogram was Aₙ. P(MT_{f,e}|Aₙ) can, for example, be modeled as follows:

P(MT_{f,e}|Aₙ)=cdf(MT_{f,e},mean=A_{n,f,e},sd=σ_{f,e})*[1−cdf(MT_{f,e},mean=A_{n,f,e},sd=σ_{f,e})].

In this equation, cdf[MT_{f,e}, mean=A_{n,f,e}, sd=σ_{f,e}] is the value of the cumulative normal distribution function with mean A_{n,f,e} (the value in dB HL of Aₙ at frequency f and in ear e) and standard deviation σ_{f,e} in dB at MT_{f,e}. Given user data from a sufficiently large group of users for whom clinical air-conduction pure-tone hearing thresholds are known, the chosen frequency and ear combination of meta thresholds MT_{f,e} and the values of parameters k and σ_{f,e} can be optimized by minimizing predicted mean squared errors, maximizing predicted Lin's concordance correlation coefficients, minimizing predicted absolute bias, and/or minimizing a linear combination of these three predicted performance metrics. Predicted performance metrics can be calculated by means of cross-validation and/or bootstrap simulation techniques. Values of k and σ_{f,e} could, for example, be: k=1000; σ_{f=1000,e=L}=20; σ_{f=2000,e=R}=10; σ_{f=4000,e=R}=15; σ_{f=6000,e=L}=12.

In some embodiments, the value of the cluster-size parameter k can vary across the different age and gender subgroups.

The addition of processing stage 802-2 to estimation model 404 can, in some examples, reduce the number of input-module tone detection threshold measurements required for reaching a certain accuracy and precision of the estimated audiogram, as this stage exploits probabilistic effects of age and gender as well as probabilistic interfrequency and interaural audiometric relationships in human audiograms.

In some examples, the auditory measurements in processing stage 802-1 of estimation module 404 are aided auditory measurements, in which the user is already fitted with a hearing device prior to the auditory measurements being performed on the user. In these examples, additional question items in processing stage 802-1 may evaluate aided hearing ability. An aided hearing profile is estimated based on the auditory measurements and/or question items (and, in some examples, device queries as described herein). In a further stage, this aided hearing profile can be used to adjust (e.g., fine-tune) one or more parameters of the hearing device. This can be applied iteratively to optimize hearing device settings for the individual user.

As mentioned, one or more additional processing stages may be included in estimation module 404. For example, an additional processing stage (referred to herein as stage C) may classify the user as having a hearing loss of a given threshold severity or not by calculating a summary statistic for the estimated audiogram of stage B and comparing this summary statistic with a decision threshold. The summary statistic can, for example, be an average of the estimated audiogram's pure-tone thresholds across a set of frequencies in both ears (referred to herein as a pure-tone average). The optimal set of frequencies included in the summary statistic as well as the optimal decision threshold can be calculated based on Receiver Operating Characteristics (ROC) applied to user data from a sufficiently large group of users for whom clinical air-conduction pure-tone hearing thresholds are known. Using an ROC decision threshold ensures an optimal trade-off between test sensitivity and specificity, i.e., as many people with hearing loss as possible are classified as having a hearing loss (high test sensitivity) and referred to a hearing care professional for further testing, while as many people without hearing loss as possible are classified as not having a hearing loss (high test specificity) and are not referred to a hearing care professional.

In some embodiments, the summary statistic can be calculated independently for each ear, in which case stage C returns a hearing-loss classification for each ear.

In some embodiments, the threshold hearing-loss severity can be pre-specified, e.g., by a hearing care professional. The threshold severity can, for example, be pre-specified in terms of a pure tone average or in terms of a standard audiogram.

In some embodiments, the estimation module may further include an additional processing stage, referred to herein as stage D. Stage D takes the estimated audiogram and hearing-loss classification from stage C as inputs and generates personalized recommendations for compensatory strategies such as effective coping behaviors or, if applicable, uptake and usage of hearing devices. For example, prospective benefits of hearing devices could be estimated based on the estimated audiogram and hearing-loss classification from stage C. Furthermore, stage D could estimate the device configuration, in terms of hardware and software programming, that best suits the user's needs. For example, Stage D can use the estimated audiogram to calculate parameter presets for hearing aids.

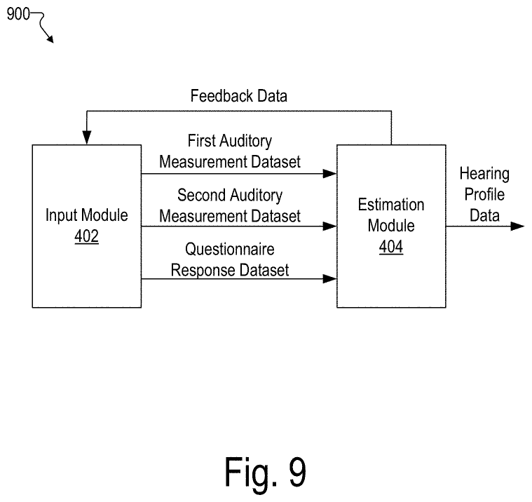

FIG. 9 shows a configuration 900 in which feedback data (e.g., the hearing profile data) generated by estimation module 404 is provided to input module 402 to determine one or more parameters used by input module 402 to acquire the auditory measurement datasets and/or the questionnaire response dataset. In this manner, estimation module 404 may adaptively influence input module 402 via a feedback loop.

For example, this adaptive hearing evaluation may be implemented by adopting a principle of minimum estimated expected entropy, whereby the auditory measurements and question items administered to the user by input module 402, which are used as input variables to a multivariate regression stage of estimation module 404, are chosen adaptively to maximize information gain on each user-input step by minimizing the estimated expected entropy of the normalized (or unnormalized) posterior probability distribution across all audiogram prototypes Aₙ.

The normalized posterior probability distribution is: P(Aₙ|SMT)=Pᵤ(Aₙ|SMT)/[ΣₙPᵤ(Aₙ|SMT)], where SMT is the set of predicted meta thresholds.

The entropy H of the normalized posterior probability distribution is: H=−ΣₙP(Aₙ|SMT)*log₂[P(Aₙ|SMT)].

For example, in an initial user-input step 1, input module 402 may administer an auditory tone detection threshold measurement at 4 kHz in the right ear and question items 1, 2, and 3, yielding the meta threshold at 4 kHz estimated by estimation module 404:

MT_{f,4000,e=R}=c_{4,0}+c_{4,1}*IMmeas_{f=4000,e=R\hat{ }}p_{4,1}+c_{4,2}*IMitem₁\hat{ }p_{4,2}+c_{4,3}*IMitem₃\hat{ }p_{4,3}.

The posterior probability P(Aₙ|SMT₁) estimated by the estimation module 404 in Error! Reference source not found., where SMT₁ represents the set of meta thresholds available after user-input step 1: SMT₁={MT_{f=4000,e=R}}.

In preparation for the next user-input step, the estimation module 404 evaluates which additional tone detection threshold measurement (e.g., 2 kHz in the right ear, 1 kHz in the left ear, or 6 kHz in the left ear) will minimize the expected entropy after the next user-input step. In order to do so, for each audiogram prototype Aₙ, estimation module 404 computes sets of conditional posterior probabilities that represent audiogram prototype Aₙ's probability contingent on a hypothetical tone detection threshold outcome IMmeas_{f,e}^{G}. The index “G” here indicates that this tone detection threshold is not the result of an actual measurement but rather one possible value on a grid of N_{G} possible outcome values, e.g., all 5-dB equidistant values from −10 to 110 dB HL. This results in N_{G} hypothetical meta thresholds at each frequency:

MT^{G}_{f=1000,e=L}=c_{1,0}+c_{1,1}*IMmeas^{G}_{f=1000,e=L}\hat{ }p_{1,1}+c_{1,2}*IMitem₁\hat{ }p_{1,2}+c_{1,3}*IMitem₂\hat{ }p_{1,3},

MT^{G}_{f=2000,e=R}=c_{2,0}+c_{2,1}*IMmeas^{G}_{f=1000,e=R}\hat{ }p_{2,1}+c_{2,2}*IMitem₁\hat{ }p_{2,2}+c_{2,3}*IMitem₃\hat{ }p_{2,3},

MT^{G}_{f=6000,e=L}=c_{6,0}+c_{6,1}*IMmeas^{G}_{f=6000,e=L}\hat{ }p_{6,1}+c_{6,2}*IMitem₁\hat{ }p_{6,2}+c_{6,3}*IMitem₃\hat{ }p_{6,3},

each with a corresponding conditional posterior probability P(Aₙ|SMT₁ U MT^{G}_{f,e}), where “U” represents the union operator. These conditional probabilities are used to define conditional entropies contingent on the measurement outcome IMmeas_{f,e}^{G}: H^{G}_{f,e}=−ΣₙP(Aₙ|SMT₁ U MT^{G}_{f,e})*log₂[P(Aₙ|SMT₁ U MT^{G}_{f,e})].

After user-input step 1, the probability of observing the meta threshold MT^{G}_{f,e} can be estimated as the weighted mean: P₁(MT^{G}_{f,e})=Σₙ P(MT^{G}_{f,e}|Aₙ)*P(Aₙ|SMT₁).

These estimated probabilities of observing meta thresholds MT^{G}_{f,e} are combined with the conditional entropies to estimate the expected entropy defined as: H_{f,e}=Σ_{G}P₁(MT^{G}_{f,e})*H^{G}_{f,e}.

Next, estimation module 404 may determine the frequency f and ear e that yields the minimal expected entropy H_{f,e}. This frequency and ear indicate the corresponding auditory tone detection threshold measurement to be administered by input module 402 in user-input step 2. This procedure can be iterated until one of two possible stopping criterions is reached, either a predefined maximum number of user-input steps or a minimum value of the entropy H of the normalized posterior probability distribution is reached.

This procedure is not limited to adaptively evaluating input-module auditory measurements. It can also be used to evaluate the information gain from presenting additional question items to the user instead of additional auditory measurements in subsequent user-input steps. In the case of evaluating a question item, the grid of N_{G} possible outcome values takes the values of the possible responses to that question item, and the frequency and ear indices f and e are replaced by an appropriate index uniquely identifying that question item.

In order to be able to predict meta thresholds for all combinations of predictor variables under consideration, regression parameters c_{n,k} and p_{n,k} may be determined by fitting regression models to user data from a sufficiently large group of users, for whom air-conduction pure-tone hearing thresholds are known and who have completed all auditory measurements and question items under consideration.

By adaptively selecting the auditory measurements and question items administered to the user, the adaptive self-administered hearing evaluation method shown in FIG. 9 maximizes information gain and results in an efficient estimation of a user's hearing profile. For example, this adaptive method may select auditory measurements from a pool of possible threshold and supra-threshold measurements and question items from a pool of various question items.

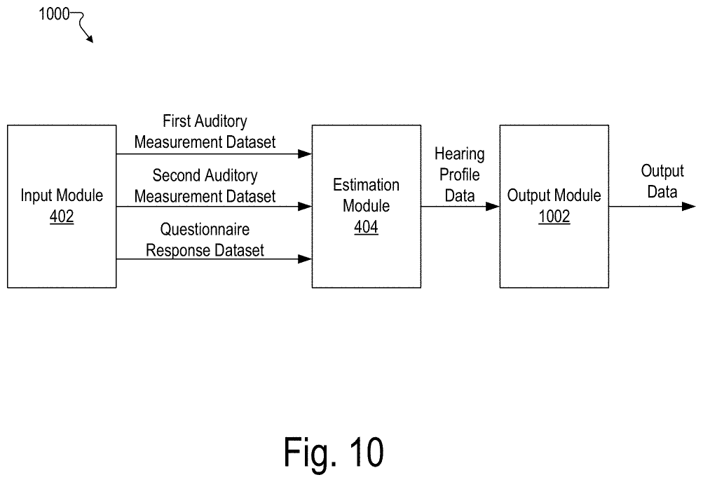

FIG. 10 shows a configuration 1000 in which system 100 further includes an output module 1002 configured to generate output data based on the hearing profile data output by estimation module 404.

The output data may, for example, include data configured to direct a display device to display information associated with the hearing profile within a graphical user interface. Such information may include a graph of the hearing profile, one or more parameter and/or statistics associated with the hearing profile, one or more characteristics of the user, etc.

In some examples, the output data may direct a display device to display high-level classification (e.g., “Congratulations. You passed this hearing evaluation.” or “You failed this hearing evaluation. Please refer for further testing.”) to the user, while also directing a different display device to display more detailed information to a hearing care professional (e.g., a graph of the hearing profile, a hearing-loss classification, and/or recommendations for compensatory strategies).

Additionally or alternatively, the output data generated by output module 1002 may include data that is to be transmitted to remote evaluation system 302, a computing device associated with a hearing care professional, and/or any other computing device or system as may serve a particular implementation. System 100 may transmit such data in any suitable manner.

Additionally or alternatively, the output data generated by output module 1002 may include data representative of one or more recommendations for the user. For example, the output data may be representative of one or more recommendations for compensatory strategies such as effective coping behaviors and usage of a hearing device.

Additionally or alternatively, the output data generated by output module 1002 may include data representative of one or more programming instructions for a hearing device being used or to be used by the user. The programming instructions may be used to program one or more parameters of the hearing device.

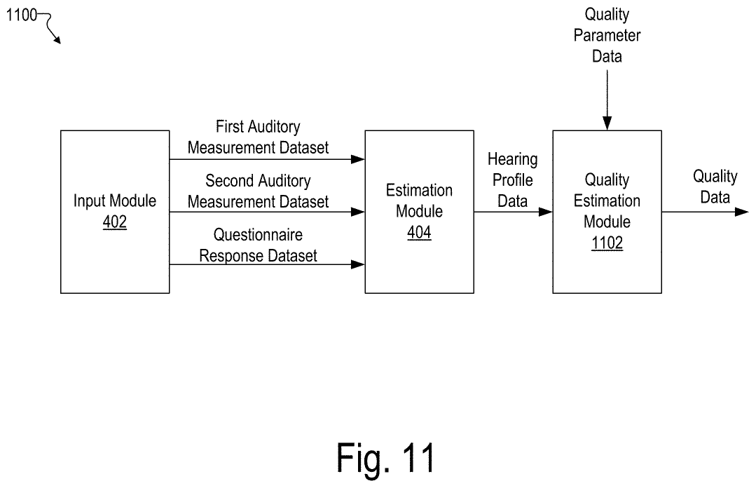

FIG. 11 shows a configuration 1100 in which system 100 further includes a quality estimation module 1102 configured to estimate a quality of the hearing profile represented by the hearing profile data. While not shown in FIG. 11, output module 1002 may be included in some implementations of configuration 1100.

As shown, quality estimation module 1102 may receive the hearing profile data output by estimation module 404 as an input. Quality estimation module 1102 may further receive quality parameter data representative of one or more parameters that may be used by quality estimation module 1102 to estimate the quality of the hearing profile. As shown, quality estimation module 1102 may output quality data indicating a quality metric of the hearing profile.

Quality estimation module 1102 may estimate the quality of the hearing profile in any suitable manner. For example, quality estimation module 1102 may determine a maximum unnormalized (or normalized) posterior value max[Pᵤ(Aₙ|SMT)] associated with the determining of the hearing profile. This value may be compared to a posterior threshold value indicating low estimating quality. Based on the comparison, quality estimation module 1102 may output quality data indicating a quality metric of the hearing profile. In some examples, the mean squared error of the estimated audiogram decreases with increasing maximum unnormalized posterior value.

In some examples, the maximum unnormalized posterior value may be passed to output module 1002, which may present to the user and/or a hearing care professional information indicating the potential low quality of the estimated hearing profile if the maximum unnormalized posterior value falls below a posterior threshold value indicating low estimation quality. This posterior threshold value can be determined by reviewing estimation model performance for a large group of users for whom clinical air-conduction pure-tone hearing thresholds are known. The posterior threshold value can, for example, be chosen such that the maximum unnormalized posterior values for a certain lower percentile of all users in the reviewed user group fall below the posterior threshold value.

One or more other types of quality parameter data may be used by quality estimation module 1102 to estimate a quality of a hearing profile output by estimation module 404. For example, quality estimation module 1102 may obtain a device dataset representative of one or more attributes of one or more devices used to perform the auditory measurements for the first and second ears of the user, an ambient noise dataset representative of one or more ambient noise levels present in an environment of the user while the auditory measurements for the first and second ears are acquired, and/or a contextual information dataset representative of one or more contextual attributes (e.g., information about the user's performance of the auditory measurements and the user's responses to the question items, such as user response times, number of user interactions, user listening duration, etc.). One or more of these datasets may be used by quality estimation module 1102 to estimate the quality of the hearing profile.

Additionally or alternatively, quality estimation module 1102 may estimate the quality of the hearing profile based on the asymmetry of auditory measurement results in the left and right ear. For example, the more asymmetric tone thresholds are in the left and right ear, the less reliable the estimated audiogram may be. In some examples, the calculation of asymmetry between the two ears does not necessitate that the thresholds are measured at the same frequencies in the two ears. The measured thresholds can instead be interpolated to a common set of frequencies in the two ears and the interpolated values can be compared to calculate asymmetry.

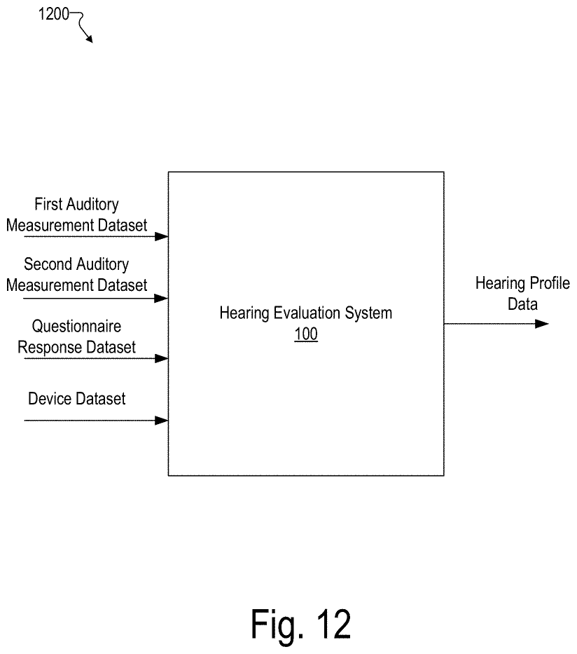

One or more additional types of datasets may be used by system 100 to determine a hearing profile for user. For example, FIG. 12 shows a configuration 1200 in which system 100 obtains a device dataset and uses the device dataset (together with the auditory measurement datasets and the questionnaire response dataset) to determine the hearing profile for the user. For example, the device dataset may be provided as an additional input to estimation module 404. The device dataset may be representative of one or more attributes of one or more devices used to perform the auditory measurements for the first and second ears of the user.

System 100 (e.g., input module 402) may obtain the device dataset in any suitable manner. For example, system 100 may query the one or more devices for the one or more attributes and receive, based on the querying, data representative of the one or more attributes from the one or more devices. Such attributes may include, but are not limited to, information indicating a manufacturer and model of the one or more devices, a device category (e.g., mobile phone, tablet, type of audio transducer, etc.) of the one or more devices, and/or any other information associated with the one or more devices. Such information can account for spectral and sound-level differences across devices and transducers.

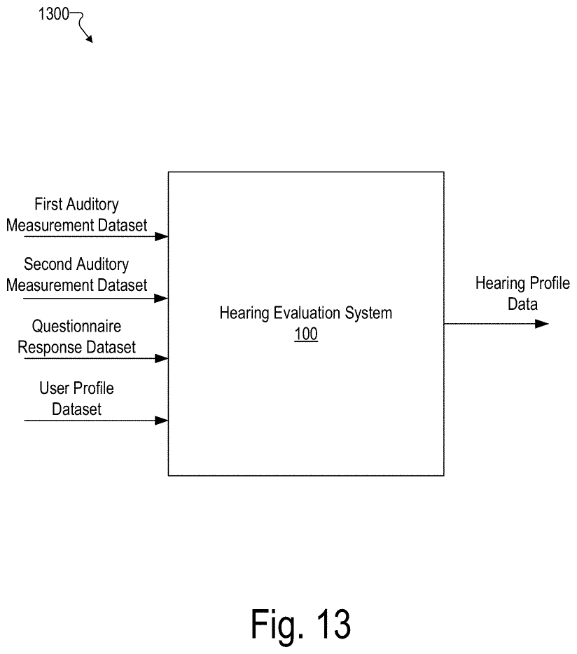

As another example, FIG. 13 shows a configuration 1300 in which system 100 obtains a user profile dataset and uses the user profile dataset (together with the auditory measurement datasets, the questionnaire response dataset, and/or the device dataset) to determine the hearing profile for the user. For example, the user profile dataset may be provided as an additional input to estimation module 404. The user profile dataset may be representative of one or more characteristics of the user. The user profile dataset may additionally be representative of one or more characteristics of a plurality of users other than the user, as described herein.

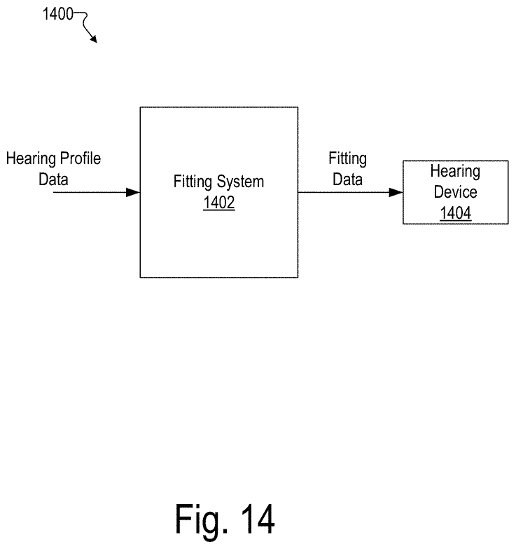

FIG. 14 shows a configuration 1400 in which a fitting system 1402 is configured to use the hearing profile data to generate fitting data that may be used to fit a hearing device 1404 to the user. Fitting system 1402 may be implemented by one or more computing devices communicatively coupled to system 100. Alternatively, fitting system 1402 may be integrated into system 100. The fitting data generated by fitting system 1402 may be configured to adjust one or more parameters of and/or otherwise program hearing device 1404.

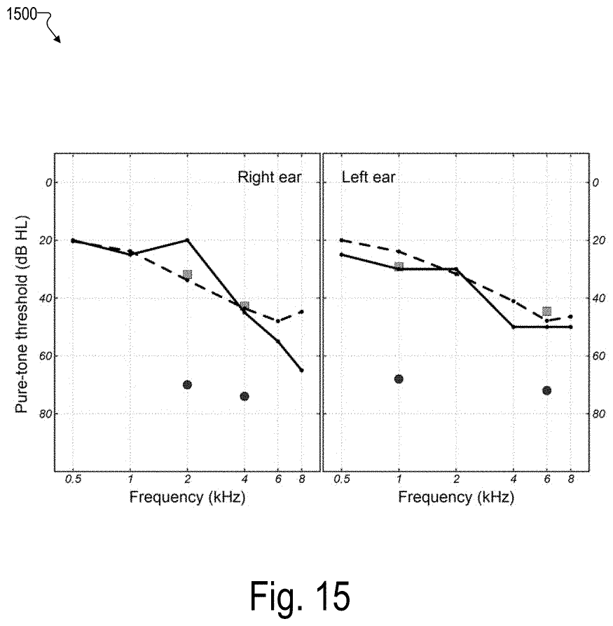

FIG. 15 shows a graph 1500 that shows how the systems and methods described herein may be used to determine a hearing profile for a user. In this example, the user was asked three questions to generate the questionnaire response dataset. These questions and the user's answers are listed in Table 1.

TABLE 1

| Question | Answer |
|---|---|
| Do you feel that you have hearing issues? | No |
| Do you find it hard to have a conversation | Rarely |
| on the phone? | |
| Do you find it hard to hear high-pitched | Never |
| sounds like bird songs? | |

In graph 1500, the solid curves show the user's clinical air-conduction audiogram (i.e., an audiogram determined by a hearing care professional in a clinic). The circular bullet symbols show the four input-module tone threshold measurements (IMmeas_{f=1000,e=L}, IMmeas_{f=2000,e=R}, IMmeas_{f=4000,e=R}, IMmeas_{f=6000,e=L}), which overshoot the air-conduction pure-tone thresholds for unknown reasons. The square symbols show the meta thresholds computed by stage A (i.e., processing stage 802-1) of estimation module 404 (MT_{f=1000,e=L}, MT_{f=2000,e=R}, MT_{f=4000,e=R}, MT_{f=6000,e=L}). As illustrated, these meta thresholds have been shifted upward toward lower thresholds due to the user's responses to the question items, which indicated that hearing difficulties were experienced rarely to never. Stage B (i.e., processing stage 802-2) of estimation module 404 integrates the meta thresholds across frequency and ears to produce the estimated audiogram represented by the dashed curves. As shown, the dashed curves closely resemble the clinical air-conduction audiogram.

In the example of FIG. 15, the auditory measurement test procedure for the left ear included the application of two acoustic stimuli each having a distinct frequency. Likewise, the auditory measurement test procedure for the right ear included the application of two acoustic stimuli each having a distinct frequency, where the distinct frequencies used for the right ear were different than the distinct frequencies used for the left ear. Both sets of distinct frequencies were included in an entire evaluation frequency range (e.g., 0.5 to 8 kHz). Based on user responses to these discrete frequencies and using the concepts described herein, estimation module 404 was able to generate a hearing profile for the entire evaluation frequency range.

While two discrete frequencies were used for each auditory measurement test procedure in FIG. 15, any number of discrete frequencies may be used for each auditory measurement test procedure as long as the total number of discrete frequencies for each auditory measurement test procedure is less than a total number of discrete frequencies included in the evaluation frequency range. As an example, the entire evaluation frequency range may include at least one thousand discrete frequencies, while the first and second discrete frequency sets each include no more than ten discrete frequencies.

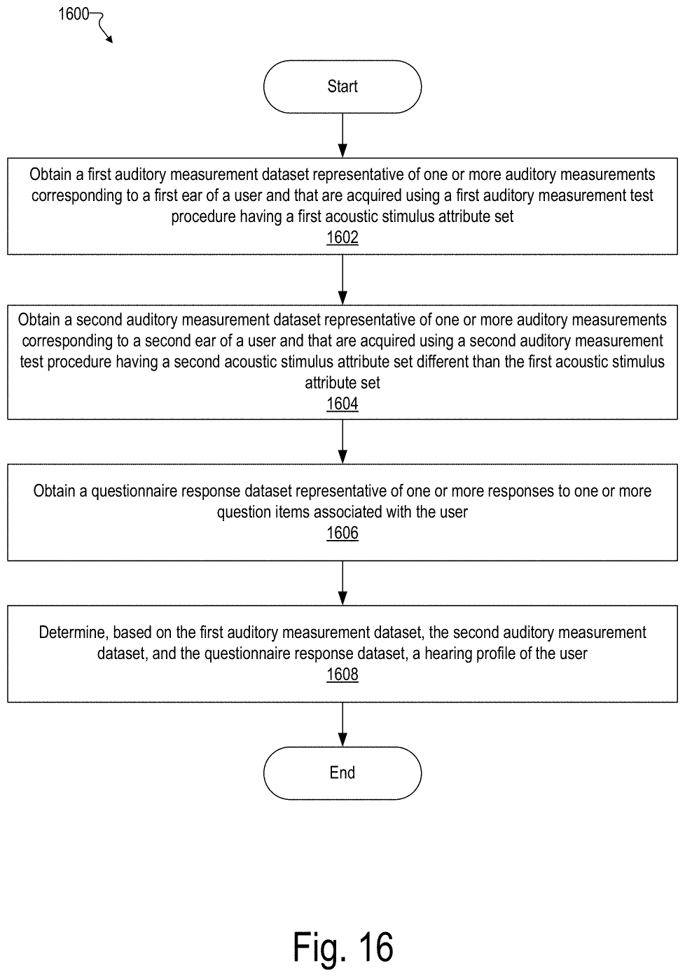

FIG. 16 illustrates an exemplary method 1600 that may be performed by system 100. While FIG. 16 illustrates exemplary operations according to one embodiment, other embodiments may omit, add to, reorder, and/or modify one or more operations of the method 1600 depicted in FIG. 16. Each operation of the method 1600 depicted in FIG. 16 may be performed in any manner described herein.

At operation 1602, a hearing evaluation system obtains a first auditory measurement dataset representative of one or more auditory measurements corresponding to a first ear of a user and that are acquired using a first auditory measurement test procedure having a first acoustic stimulus attribute set.

At operation 1604, the hearing evaluation system obtains a second auditory measurement dataset representative of one or more auditory measurements corresponding to a second ear of a user and that are acquired using a second auditory measurement test procedure having a second acoustic stimulus attribute set different than the first acoustic stimulus attribute set.

At operation 1606, the hearing evaluation system obtains a questionnaire response dataset representative of one or more responses to one or more question items associated with the user.

At operation 1608, the hearing evaluation system determines, based on the first auditory measurement dataset, the second auditory measurement dataset, and the questionnaire response dataset, a hearing profile of the user.

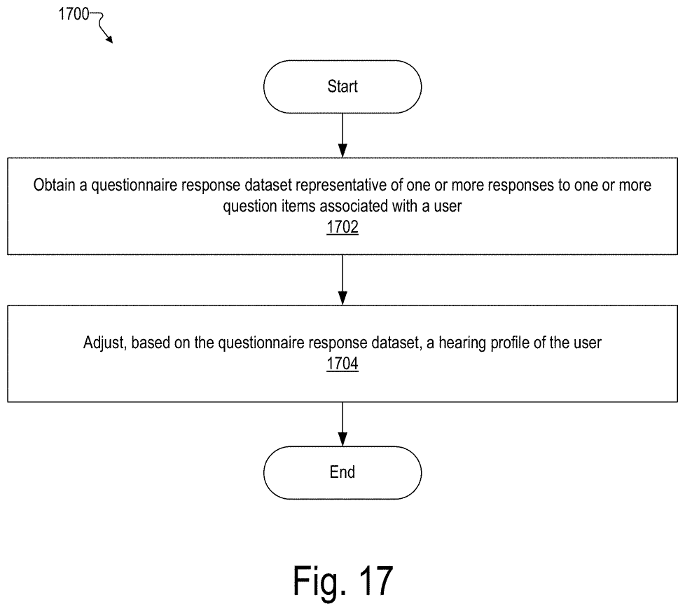

FIG. 17 illustrates another exemplary method 1700 that may be performed by system 100. While FIG. 17 illustrates exemplary operations according to one embodiment, other embodiments may omit, add to, reorder, and/or modify one or more operations of the method 1700 depicted in FIG. 17. Each operation of the method 1700 depicted in FIG. 17 may be performed in any manner described herein.

At operation 1702, a hearing evaluation system obtains a questionnaire response dataset representative of one or more responses to one or more question items associated with a user.

At operation 1704, the hearing evaluation system adjusts, based on the questionnaire response dataset, a hearing profile of the user.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

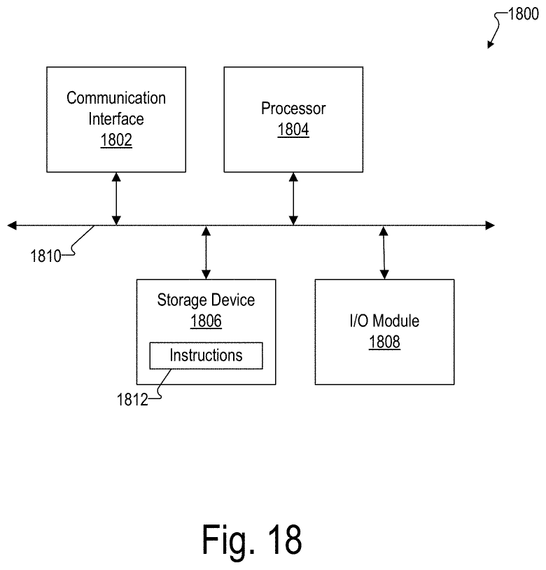

FIG. 18 illustrates an exemplary computing device 1800 that may be specifically configured to perform one or more of the processes described herein. To that end, any of the systems, processing units, and/or devices described herein may be implemented by computing device 1800.

As shown in FIG. 18, computing device 1800 may include a communication interface 1802, a processor 1804, a storage device 1806, and an input/output (“I/O”) module 1808 communicatively connected one to another via a communication infrastructure 1810. While an exemplary computing device 1800 is shown in FIG. 18, the components illustrated in FIG. 18 are not intended to be limiting. Additional or alternative components may be used in other embodiments. Components of computing device 1800 shown in FIG. 18 will now be described in additional detail.

Communication interface 1802 may be configured to communicate with one or more computing devices. Examples of communication interface 1802 include, without limitation, a wired network interface (such as a network interface card), a wireless network interface (such as a wireless network interface card), a modem, an audio/video connection, and any other suitable interface.

Processor 1804 generally represents any type or form of processing unit capable of processing data and/or interpreting, executing, and/or directing execution of one or more of the instructions, processes, and/or operations described herein. Processor 1804 may perform operations by executing computer-executable instructions 1812 (e.g., an application, software, code, and/or other executable data instance) stored in storage device 1806.

Storage device 1806 may include one or more data storage media, devices, or configurations and may employ any type, form, and combination of data storage media and/or device. For example, storage device 1806 may include, but is not limited to, any combination of the non-volatile media and/or volatile media described herein. Electronic data, including data described herein, may be temporarily and/or permanently stored in storage device 1806. For example, data representative of computer-executable instructions 1812 configured to direct processor 1804 to perform any of the operations described herein may be stored within storage device 1806. In some examples, data may be arranged in one or more databases residing within storage device 1806.

I/O module 1808 may include one or more I/O modules configured to receive user input and provide user output. I/O module 1808 may include any hardware, firmware, software, or combination thereof supportive of input and output capabilities. For example, I/O module 1808 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1808 is configured to provide graphical data to a display for presentation to a user. The graphical data may be representative of one or more graphical user interfaces and/or any other graphical content as may serve a particular implementation.

Advantages and features of the present disclosure can be further described by the following statements.

1. A system comprising: a memory storing instructions; a processor communicatively coupled to the memory and configured to execute the instructions to: obtain a first auditory measurement dataset representative of one or more auditory measurements corresponding to a first ear of a user and that are acquired using a first auditory measurement test procedure having a first acoustic stimulus attribute set, obtain a second auditory measurement dataset representative of one or more auditory measurements corresponding to a second ear of the user and that are acquired using a second auditory measurement test procedure having a second acoustic stimulus attribute set different than the first acoustic stimulus attribute set, obtain a questionnaire response dataset representative of one or more responses to one or more question items associated with the user, and determine, based on the first auditory measurement dataset, the second auditory measurement dataset, and the questionnaire response dataset, a hearing profile of the user.

2. The system of any of the preceding statements, wherein: the first acoustic stimulus attribute set comprises a first discrete frequency of a first acoustic stimulus presented to the user during the first auditory measurement test procedure; the second acoustic stimulus attribute set comprises a second discrete frequency of a second acoustic stimulus presented to the user during the second auditory measurement test procedure; the second discrete frequency is different than the first discrete frequency.

3. The system of any of the preceding statements, wherein: the first discrete frequency is included in a first discrete frequency set corresponding to the first auditory measurement dataset, the first discrete frequency set including less than a total number of discrete frequencies in an entire evaluation frequency range and not including the second discrete frequency; the second discrete frequency is included in a second discrete frequency set corresponding to the second auditory measurement dataset, the second discrete frequency set including less than the total number of discrete frequencies in the entire evaluation frequency range; and the hearing profile corresponds to the entire evaluation frequency range.

4. The system of any of the preceding statements, wherein: the entire evaluation frequency range includes at least one thousand discrete frequencies; and the first and second discrete frequency sets each include no more than ten discrete frequencies.

5. The system of any of the preceding statements, wherein the first and second discrete frequency sets each include only two discrete frequencies.

6. The system of any of the preceding statements, wherein: the first acoustic stimulus attribute set comprises one or more attributes representative of one or more frequencies, spectral characteristics, temporal characteristics, or perceptive attributes of one or more acoustic stimuli used during the first auditory measurement test procedure; and the second acoustic stimulus attribute set comprises one or more attributes representative of one or more frequencies, temporal characteristics, or perceptive attributes of one or more acoustic stimuli used during the second auditory measurement test procedure; wherein at least one attribute of the one or more attributes included in the second acoustic stimulus attribute set is not included in the first acoustic stimulus attribute set.

7. The system of any of the preceding statements, wherein the obtaining of the first auditory measurement dataset comprises: directing an acoustic signal generator to present an auditory stimulus to the first ear of the user, the auditory stimulus having an attribute included in the first acoustic stimulus attribute set; and measuring a response by the user to the auditory stimulus.

8. The system of any of the preceding statements, wherein the directing of the acoustic signal generator to present the auditory stimulus to the first ear of the user comprises transmitting, by way of a network, a command to a computing system located within a user premises of the user for the computing system to present the auditory stimulus to the first ear of the user.

9. The system of any of the preceding statements, wherein the measuring comprises one or more of receiving manual user input representative of the response, detecting a verbal response from the user, using pupillometry to measure the response, recording a video of a physical response by the user to the auditory stimulus, measuring a biopotential response to the auditory stimulus, recording a conductance of the user's skin, detecting an optical response to the auditory stimulus with an optical sensor, or detecting a mechanical response to the auditory stimulus with a mechanical sensor.

10. The system of any of the preceding statements, wherein the determining of the hearing profile comprises: applying the first auditory measurement dataset, the second auditory measurement dataset, and the questionnaire response dataset as inputs to a predictive model; and generating the hearing profile based on an output of the predictive model.

11. The system of any of the preceding statements, wherein the predictive model comprises a multivariate regression model that uses one or more of linear regression or non-linear regression.

12. The system of any of the preceding statements, wherein the output of the multivariate regression model is based on a fitting of one or more regression models to historical user data corresponding to a plurality of users other than the user, the historical user data representative of auditory measurements performed on the plurality of users.

13. The system of any of the preceding statements, wherein the predictive model comprises a machine learning model.

14. The system of any of the preceding statements, wherein the machine learning model is trained based on historical user data corresponding to a plurality of users other than the user, the historical user data representative of auditory measurements performed on the plurality of users.

15. The system of any of the preceding statements, wherein the historical user data is further representative of one or more responses to one or more question items associated with the plurality of users.

16. The system of any of the preceding statements, wherein the machine learning model is implemented by a supervised deep learning model.

17. The system of any of the preceding statements, wherein the processor is further configured to estimate a quality of the hearing profile.

18. The system of any of the preceding statements, wherein the estimating of the quality of the hearing profile comprises: determining a maximum posterior value associated with the determining of the hearing profile; and comparing the maximum posterior value to a posterior threshold value indicating low estimating quality.

19. The system of any of the preceding statements, wherein the estimating of the quality of the hearing profile comprises: obtaining a device dataset representative of one or more attributes of one or more devices used to perform the auditory measurements for the first and second ears of the user; and using the device dataset to estimate the quality of the hearing profile.

20. The system of any of the preceding statements, wherein the estimating of the quality of the hearing profile comprises: obtaining an ambient noise dataset representative of one or more ambient noise levels present in an environment of the user while the auditory measurements for the first and second ears are acquired; and using the ambient noise dataset to estimate the quality of the hearing profile.

21. The system of any of the preceding statements, wherein the estimating of the quality of the hearing profile comprises: obtaining a contextual information dataset representative of one or more contextual attributes of one or more of the first auditory measurement dataset, the second auditory measurement dataset, or the questionnaire response dataset; and using the contextual information dataset to estimate the quality of the hearing profile.

22. The system of any of the preceding statements, wherein the processor is further configured to execute the instructions to use data representative of the hearing profile to determine one or more parameters used to acquire one or more of the auditory measurement dataset, the second auditory measurement dataset, or the questionnaire response dataset.

23. The system of any of the preceding statements, wherein the processor is further configured to execute the instructions to: determine a summary statistic for the hearing profile; and determine, based on a comparison of the summary statistic with a decision threshold, a degree of hearing loss for the user.

24. The system of any of the preceding statements, wherein the processor is further configured to execute the instructions to generate, based on the degree of hearing loss for the user, one or more personalized recommendations for the user to cope with the hearing loss.

25. The system of any of the preceding statements, wherein: the user is already fitted with a hearing device prior to the auditory measurements for the first and second ears being performed on the user; the user uses the hearing device during while the auditory measurements for the first and second ears are performed on the user; and the hearing profile represents an aided hearing profile representative of a hearing ability of the user while the user uses the hearing device.

26. The system of any of the preceding statements, wherein the processor is further configured to execute the instructions to adjust one or more parameters of the hearing device based on the aided hearing profile.

27. The system of any of the preceding statements, wherein the processor is further configured to execute the instructions to fit, based on the hearing profile, a hearing device to the user.

28. The system of any of the preceding statements, wherein the processor is further configured to execute the instructions to present a graph of the hearing profile within a graphical user interface.

29. The system of any of the preceding statements, wherein: the processor is further configured to obtain a device dataset representative of one or more attributes of one or more devices used to perform the auditory measurements for the first and second ears of the user; and the determining of the hearing profile is further based on the device dataset.

30. The system of any of the preceding statements, wherein the obtaining of the device dataset comprises: querying the one or more devices for the one or more attributes of the one or more devices; and receiving, based on the querying, data representative of the one or more attributes from the one or more devices.

31. The system of any of the preceding statements, wherein: the processor is further configured to obtain a user profile dataset representative of one or more characteristics of the user; and the determining of the hearing profile is further based on the user profile dataset.

32. The system of any of the preceding statements, wherein the determining of the hearing profile is further based on a user profile dataset representative of one or more characteristics of a plurality of users other than the user.

33. The system of any of the preceding statements, wherein: the first auditory measurement dataset, the second auditory measurement dataset, and the questionnaire response dataset are acquired by one or more computing devices physically located within a user premises while the user is also located within a user premises; and the obtaining of the first auditory measurement dataset, the second auditory measurement dataset, and the questionnaire response dataset comprises receiving, by way of a network, the first auditory measurement dataset, the second auditory measurement dataset, and the questionnaire response dataset from the one or more computing devices.

34. The system of any of the preceding statements, wherein the obtaining of the questionnaire response dataset comprises one or more of receiving user input representative of one or more of the responses from the user or receiving user input representative of one or more of the responses from a person other than the user.

35. The system of any of the preceding statements, wherein the hearing profile comprises one or more of an audiogram for the user or a profile that indicates a likelihood of hearing loss or hearing impairment in the user.

36. The system of any of the preceding statements, wherein the hearing profile is representative of a binaural hearing capability of the user.

37. A system comprising: a memory storing instructions; a processor communicatively coupled to the memory and configured to execute the instructions to: obtain a questionnaire response dataset representative of one or more responses to one or more question items associated with a user, and adjust, based on the questionnaire response dataset, a hearing profile of the user.

38. The system of statement 37, wherein the processor is further configured to execute the instructions to: access data representative of a predictive model; wherein the adjusting of the hearing profile is further based on the predictive model.

39. A method implementing any of the operations of the preceding statements.

40. A non-transitory computer-readable medium storing instructions that, when executed, direct a processor of a computing device to perform any of the operations of the preceding statements.

41. A system comprising: a memory storing instructions; a processor communicatively coupled to the memory and configured to execute the instructions to: obtain a first auditory measurement dataset representative of auditory measurements for a first ear of a user, the first auditory measurement dataset corresponding to a first discrete frequency set that includes less than a total number of discrete frequencies in an entire evaluation frequency range, obtain a second auditory measurement dataset representative of auditory measurements for a second ear of the user, the second auditory measurement dataset corresponding to a second discrete frequency set that includes less than the total number of discrete frequencies in the entire evaluation frequency range, the second discrete frequency set including at least one discrete frequency not included in the first discrete frequency set, obtain a questionnaire response dataset representative of one or more responses to one or more question items associated with the user, and determine, based on the first auditory measurement dataset, the second auditory measurement dataset, and the questionnaire response dataset, a hearing profile for the user and that corresponds to the entire evaluation frequency range.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
a memory storing instructions; and
a processor communicatively coupled to the memory and configured to execute the instructions to:
obtain a first auditory measurement dataset representative of one or more auditory measurements corresponding to a first ear of a user and that are acquired using a first auditory measurement test procedure having a first acoustic stimulus attribute set,
obtain a second auditory measurement dataset representative of one or more auditory measurements corresponding to a second ear of the user and that are acquired using a second auditory measurement test procedure having a second acoustic stimulus attribute set different than the first acoustic stimulus attribute set,
obtain a questionnaire response dataset representative of one or more responses to one or more question items associated with the user, and
determine, based on the first auditory measurement dataset, the second auditory measurement dataset, and the questionnaire response dataset, a hearing profile of the user.

2. The system of claim 1, wherein:
the first acoustic stimulus attribute set comprises a first discrete frequency of a first acoustic stimulus presented to the user during the first auditory measurement test procedure;
the second acoustic stimulus attribute set comprises a second discrete frequency of a second acoustic stimulus presented to the user during the second auditory measurement test procedure; and
the second discrete frequency is different than the first discrete frequency.

3. The system of claim 2, wherein:
the first discrete frequency is included in a first discrete frequency set corresponding to the first auditory measurement dataset, the first discrete frequency set including less than a total number of discrete frequencies in an entire evaluation frequency range and not including the second discrete frequency;
the second discrete frequency is included in a second discrete frequency set corresponding to the second auditory measurement dataset, the second discrete frequency set including less than the total number of discrete frequencies in the entire evaluation frequency range; and
the hearing profile corresponds to the entire evaluation frequency range.

4. The system of claim 1, wherein:
the first acoustic stimulus attribute set comprises one or more attributes representative of one or more frequencies, spectral characteristics, temporal characteristics, or perceptive attributes of one or more acoustic stimuli used during the first auditory measurement test procedure; and
the second acoustic stimulus attribute set comprises one or more attributes representative of one or more frequencies, temporal characteristics, or perceptive attributes of one or more acoustic stimuli used during the second auditory measurement test procedure;
wherein at least one attribute of the one or more attributes included in the second acoustic stimulus attribute set is not included in the first acoustic stimulus attribute set.

5. The system of claim 1, wherein the obtaining of the first auditory measurement dataset comprises:
directing an acoustic signal generator to present an auditory stimulus to the first ear of the user, the auditory stimulus having an attribute included in the first acoustic stimulus attribute set; and
measuring a response by the user to the auditory stimulus.

6. The system of claim 5, wherein the directing of the acoustic signal generator to present the auditory stimulus to the first ear of the user comprises transmitting, by way of a network, a command to a computing system located within a user premises of the user for the computing system to present the auditory stimulus to the first ear of the user.

7. The system of claim 1, wherein the determining of the hearing profile comprises:
applying the first auditory measurement dataset, the second auditory measurement dataset, and the questionnaire response dataset as inputs to a predictive model; and
generating the hearing profile based on an output of the predictive model.

8. The system of claim 7, wherein the predictive model comprises a multivariate regression model that uses one or more of linear regression or non-linear regression.

9. The system of claim 7, wherein the predictive model comprises a machine learning model.

10. The system of claim 1, wherein the processor is further configured to estimate a quality of the hearing profile.

11. The system of claim 10, wherein the estimating of the quality of the hearing profile comprises:
determining a maximum posterior value associated with the determining of the hearing profile; and
comparing the maximum posterior value to a posterior threshold value indicating low estimating quality.

12. The system of claim 1, wherein the processor is further configured to execute the instructions to use data representative of the hearing profile to determine one or more parameters used to acquire one or more of the auditory measurement dataset, the second auditory measurement dataset, or the questionnaire response dataset.

13. The system of claim 1, wherein the processor is further configured to execute the instructions to:
determine a summary statistic for the hearing profile; and
determine, based on a comparison of the summary statistic with a decision threshold, a degree of hearing loss for the user.

14. The system of claim 1, wherein the obtaining of the questionnaire response dataset comprises one or more of receiving user input representative of one or more of the responses from the user or receiving user input representative of one or more of the responses from a person other than the user.

15. The system of claim 1, wherein the processor is further configured to execute the instructions to fit, based on the hearing profile, a hearing device to the user.

16. The system of claim 1, wherein:
the user is already fitted with a hearing device prior to the auditory measurements for the first and second ears being performed on the user;
the user uses the hearing device during while the auditory measurements for the first and second ears are performed on the user; and
the hearing profile represents an aided hearing profile representative of a hearing ability of the user while the user uses the hearing device.

17. The system of claim 1, wherein:
the processor is further configured to obtain a device dataset representative of one or more attributes of one or more devices used to perform the auditory measurements for the first and second ears of the user; and
the determining of the hearing profile is further based on the device dataset.

18. A system comprising:
a memory storing instructions; and
a processor communicatively coupled to the memory and configured to execute the instructions to:
obtain a questionnaire response dataset representative of one or more responses to one or more question items associated with a user, and
adjust, based on the questionnaire response dataset, a hearing profile of the user.

19. The system of claim 18, wherein the processor is further configured to execute the instructions to:
access data representative of a predictive model;
wherein the adjusting of the hearing profile is further based on the predictive model.

20. A method comprising:
obtaining, by a hearing evaluation system, a first auditory measurement dataset representative of one or more auditory measurements corresponding to a first ear of a user and that are acquired using a first auditory measurement test procedure having a first acoustic stimulus attribute set;
obtaining, by the hearing evaluation system, a second auditory measurement dataset representative of one or more auditory measurements corresponding to a second ear of the user and that are acquired using a second auditory measurement test procedure having a second acoustic stimulus attribute set different than the first acoustic stimulus attribute set;
obtaining, by the hearing evaluation system, a questionnaire response dataset representative of one or more responses to one or more question items associated with the user; and
determining, by the hearing evaluation system based on the first auditory measurement dataset, the second auditory measurement dataset, and the questionnaire response dataset, a hearing profile of the user.
